(19) United States
(12) Patent Application Publication
(10) Pub. No.: US 2018/0321244 A1
(43) Pub. Date: Nov. 8, 2018

(54) Method for Detection And Diagnosis of Oral Cancer in a sample

(71) Applicant: YENEPOYA UNIVERSITY, Mangalore (IN)

(72) Inventors: Riaz ABDULLA, Mangalore (IN); Rekha PUNCHAPPADY DEVASYA, Puttur (IN); Sneha PINTO MARIA, Bangalore (IN); Yashwanth Subbannayya, Manipal (IN); Johan HAFIZ ISKANDAR, Kuala Lumpur (MY); Arif ANWAR, Kuala Lumpur (MY); Farhaad YENEPOYA MOHAMMED, Mangalore (IN)

(21) Appl. No.: 15/971,570

(22) Filed: May 4, 2018

(30) Foreign Application Priority Data
May 4, 2017 (IN) 201741015794

(51) Int. Cl. G01N 33/574; C07K 16/18

(52) U.S. Cl. CPC G01N 33/574; G01N 2800/60; C07K 16/18

(57) ABSTRACT

The present disclosure relates to a method for detecting a biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1), in a sample. The present disclosure provides for the use of biomarker for the detection of oral cancer in a sample. The detection of the biomarker helps in early diagnosis of oral cancer.

### FIELD

The present disclosure relates to a method for detecting a biomarker in a sample.

### ABBREVIATIONS

NUBP2: Nucleotide binding protein 2;

TSPY3: Testis specific protein Y-linked 3;

XAGE4: XAGE-4 protein;

TSPY2: Testis specific protein Y-linked 2;

RPA2: Replication protein A2;

PYCR1: Pyrroline-5-carboxylate reductase 1;

TK1: Thymidine kinase 1;

HMGN5/NSBP1: High mobility group nucleosome binding domain 5;

ROPN1: Rhophilin associated protein 1 (Ropporin);

RBM46: RNA binding motif protein 46;

PSME3: Proteasome (prosome, macropain) activator subunit 3;

KRT19: Keratin 19;

TAGLN: Transgelin;

PRDX1: Peroxiredoxin;

S100A9: S100 Calcium binding protein A9 (calgranulin B); and

IMPDH1: Inosine monophosphate dehydrogenase 1.

### Definitions

As used in the present disclosure, the following terms are generally intended to have the meaning as set forth below, except to the extent that the context in which they are used indicate otherwise.

Array: A microarray (also commonly known as DNA chip or biochip) is a collection of microscopic spots of antigens or antibodies which are fixed on a solid surface such as glass, plastic or silicon chip, for the purpose of detecting antigens/antibodies or used to measure the expression levels of a large number of genes simultaneously or to genotype multiple regions of a genome.

Autoantibody: An autoantibody is an antibody (a type of protein) produced by the immune system that is directed against one or more of the individual's own tissues, cells, or cell components. The term “autoantibody” refers to antibodies that react with self-antigens which may comprise proteins, nucleic acids, carbohydrates, lipids or various combinations of these. An autoantibody is one that is produced by an organism in response to constituents, i.e., antigens that form a part of that organism.

Biomarker: Biomarker is a naturally occurring molecule, gene, or characteristic by which a particular pathological or physiological process, disease, etc. can be identified. It is a measurable indicator of the severity or presence of some disease, normal biological processes, pathogenic processes, or pharmacologic responses to a therapeutic intervention. More generally a biomarker is anything that can be used as an indicator of a particular disease state or some other physiological state of an organism.

Immunome: The term “immunome” refers to all the genes and proteins that are associated with the immune system.

Volcano Plot: The term “volcano plot” refers to a type of scatter-plot that is used to identify changes in large data sets composed of replicate data. It plots significance versus fold-change on the y and x axes, respectively.

Penetrance-based threshold analysis: The term “penetrance-based threshold analysis” refers to the measurement of likelihood of a given raw fold change being true, in order to increase the significance and reliability of result.

Down-regulation: The term “down-regulation” refers to the process by which a cell decreases the quantity of a cellular component, such as RNA or protein, in response to an external stimulus.

Up-regulation: The term “up-regulation” refers to the process by which a cell increases the quantity of a cellular component, such as RNA or protein, in response to an external stimulus.

Capture antibody component: The term “capture antibody component” refers a primary antibody, which is specific for a target biomarker/molecule and will specifically bind to it.

Detection antibody component: The term “detection antibody component” refers to a secondary antibody that will help in production of color in order to easily detect the presence of the target biomarker/molecule.

### BACKGROUND

Cancer is a group of diseases involving abnormal cell growth with the potential to invade or spread to other parts of the body. Oral cancer appears as a growth or sore in the mouth that does not go away. Oral cancer, which includes cancers of the lips, tongue, cheeks, floor of the mouth, hard and soft palate, sinuses, and pharynx (throat), can be life threatening if not diagnosed and treated early. From an epidemiological and clinical-pathological perspective, oral cancer can be divided into three categories: 1) carcinomas of the oral cavity proper, 2) carcinomas of the lip vermilion and 3) carcinomas arising in the oropharynx. Oral cancer tumours arise through a series of molecular mutations that lead to uncontrolled cellular growth from hyperplasia to dysplasia to carcinoma in situ followed by invasive carcinoma. Major risk factors that cause oral cancer include tobacco and alcohol consumption along with environmental and genetics factors. There are four stages of oral cancer. Stages 1 and 2 usually involve a small tumor. In these stages, cancer cells have not spread to the lymph nodes. Stages 3 and 4 are considered advanced stages of cancer. In these stages, tumors are large and the cancer cells have usually spread to the lymph nodes or other parts of the body. Oral cancers are most often discovered only after they have spread to the lymph nodes of the neck. Early detection is key to surviving oral cancer, as earlier the stage of diagnosis, the higher the chance of survival after treatment. This makes timely diagnosis all the more important. Biomarkers are measurable parameters of the human body that serve as indicators of underlying biological or pathological processes. Biomarkers are useful in a number of ways including measuring the progression of disease, evaluating the most effective therapeutic regimes for a particular cancer type and establishing long-term susceptibility to cancer or its recurrence. Biomarkers also help in the early diagnosis of disease, disease prevention, drug target identification, evaluation of drug response, etc. Despite the spectacular technological advances that have allowed the scientific community to measure an ever-expanding list of body parameters with greater sensitivity and specificity than ever before, these advances have not translated into greater numbers of clinically useful biomarkers, including those related to the measurement of immunological function/response of the body in response to a disease state.

Biomarkers guide patient management in a multitude of settings including screening, diagnosis, prognosis, treatment choice and treatment monitoring. They also serve as primary sources of efficacy and safety data required by the Food and Drug Administration for the approval of new therapies and medical devices. Despite the widespread and ever-growing need for new biomarkers and the large investments made in their development by funding agencies and industry, the failure rate for biomarker development is extraordinarily high. Despite tens of thousands of reports of putative new biomarkers in peer-reviewed literature, only a handful are qualified for drug development or approved for clinical use by the FDA and only about 100 biomarkers have proven clinically useful and reliable enough to be used in routine medical practice.

Detection of diseases such as oral cancers at an early stage is imperative as successful treatment often depends on early detection. Also, some oral premalignant and early cancerous lesions are not readily detectable by visual inspection. Therefore, the integration of early detection and screening based on protein biomarkers in conjunction with a conventional oral examination is extremely important. Protein biomarkers as detection tools are particularly suitable because they are responsive to simple blood or saliva tests and such tests are easy to administer as opposed to time consuming, difficult, costly and painful tissue biopsies that can only be performed by doctors authorized to do the same.

Therefore, there is a need for developing clinical tools and applications that comprise and utilize single and multiple protein and autoantibody biomarkers having high sensitivity and specificity.

### Objects

Some of the objects of the present disclosure, which at least one embodiment herein satisfies, are as follows:

It is an object of the present disclosure to ameliorate one or more problems of the prior art or to at least provide a useful alternative.

Another object of the present disclosure is to provide a method for detecting a biomarker in a sample.

Another object of the present disclosure is to provide single, multiple and a panel of protein and autoantibody biomarkers for diagnosing oral cancer.

Other objects and advantages of the present disclosure will be more apparent from the following description, which is not intended to limit the scope of the present disclosure.

### SUMMARY

The present disclosure provides a method for detecting at least one biomarker in a sample. The method comprises first diluting a sample contacting the at least one biomarker with serum albumin buffer to obtain a diluted sample. Contacting the diluted with a reagent comprising a set of capture antibody component and at least one detection antibody component. This is followed by the analysis of the change in the intensity of the detection antibody component to detect the presence of the biomarker and the extent of its expression. The capture antibody component is capable of binding with the biomarker in one to one, one to many, and many to many correspondences, whereas the detection antibody component is capable of detecting the binding of the capture antibody component with the biomarker.

Typically, the biomarker can be selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

The set of capture antibody component comprises antibody against at least one biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

The present disclosure also provides for the use of biomarker for the detection of oral cancer. The biomarker can be at least one selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

The early detection and the determination of the expression level of the biomarkers may help in the early diagnosis of oral cancer.

### BRIEF DESCRIPTION OF THE ACCOMPANYING DRAWING

The present disclosure will now be described with the help of the self-explanatory non-limiting accompanying drawing, in which:

FIG. 1 illustrates relative expression (up-regulation) of Thymidine kinase 1 (TK1) across oral cancer (1-20) and healthy control (21-40) samples;

wherein X-axis depicts number of patient samples and Y-axis depicts normalized Relative Fluorescence Units (RFU) values;

FIG. 2 illustrates relative expression (up-regulation) of PYCR1 (pyrroline-5-carboxylate reductase 1) across oral cancer (1-20) and healthy control (21-40) samples;

wherein X-axis depicts number of patient samples and Y-axis depicts normalized Relative Fluorescence Units (RFU) values;

FIG. 3 illustrates relative expression (up-regulation) of Proteasome activator subunit 3 (PSME3) across oral cancer (1-20) and healthy control (21-40) samples;

wherein X-axis depicts number of patient samples and Y-axis depicts normalized Relative Fluorescence Units (RFU) values;

FIG. 4 illustrates relative expression (up-regulation) of Replication protein A2 (RPA2) across oral cancer (1-20) and healthy control (21-40) samples;

wherein X-axis depicts number of patient samples and Y-axis depicts normalized Relative Fluorescence Units (RFU) values; and

FIG. 5 illustrates the Volcano Plot of the up-regulated and down-regulated biomarkers using penetrance-based analysis;

wherein X-axis depicts the overall fold change and the Y-axis depicts the p-Value of the biomarkers.

### DETAILED DESCRIPTION

Early diagnosis of oral cancer is vital, as successful treatment often depends on early detection. Moreover, some premalignant and early cancerous lesions are not readily detectable by visual inspection. Therefore, the integration of early detection and screening based on biomarkers in conjunction with a conventional oral examination is extremely important. Autoantibodies are known to be produced by patients in certain diseases such as autoimmune diseases and cardiovascular-related disorders, in some cases even before the onset of the disease. The autoantibody response to a single or combination of these biomarkers can be utilized for early detection, prognosis, and potential target for therapy. However, most diagnostic tests are based on single biomarkers. Constructing a clinical decision on a single biomarker can lead to a significant level of false positives. Antibodies (autoantibodies) have several properties which make them excellent indicators of disease. Unlike most other proteins found in serum, autoantibodies are stable, abundant, and highly specific, easily purified from serum, and are readily detected.

Due to their inherent amplification within the immune system, autoantibodies are relatively abundant and are easily measured, making them ideal for the detection of disease at an early stage when other potential biomarkers may be undetectable. Autoantibody biomarkers with high sensitivity and specificity can be identified accurately and easily utilising samples obtained with minimally invasive techniques.

In one aspect of the present disclosure, there is provided a method for detecting at least one biomarker in a sample. The method comprises the following steps:

Initially, a sample is diluted with serum albumin buffer to obtain a diluted sample. The diluted sample is then contacted with a reagent. The reagent comprises a set of capture antibody component and at least one detection antibody component.

The capture antibody component is capable of binding with at least one biomarker in one to one, one to many, and many to many correspondence. This means one capture antibody component may bind with one biomarker, or more than one biomarker. Alternatively, one biomarker may bind with one capture antibody component, or more than one capture antibody component.

Typically, the biomarker is at least one selected from the group consisting of genes or parts thereof, non-coding DNA, mRNA or other RNA molecules, proteins and so on. In an embodiment, the at least one biomarker is a protein. Typically, at least one biomarker is an autoantibody to a protein or other antigen. Typically, at least one autoantibody is detected by using at least one oral cancer biomarker.

In accordance with the embodiments of the present disclosure, the biomarker can be selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

The set of capture antibody component typically comprises antibody against at least one biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

The method of the present disclosure further comprises correlating the binding of the capture antibody component with at least one biomarker in the sample with diagnosis of oral cancer.

The method provides for the detection and measurement of single and/or multiple biomarkers in samples in order to diagnose oral cancer. The biomarkers are differentially expressed in the serum of individuals suffering from an oral disease, such as oral cancer as compared to the serum of an individual not suffering from said disease state. In an embodiment of the present disclosure, the method of diagnosing oral cancer in an individual comprises obtaining a sample (biological sample) from the individual and detecting the presence at least one, multiple or a panel of biomarker/s identified as associated with oral cancer and the screening and analysis of the at least one biomarker from the sample. In an embodiment of the present disclosure, the method of diagnosing oral cancer comprises determining and/or measuring the level of at least one biomarker.

In accordance with the embodiments of the present disclosure, the sample is at least one selected from the group consisting of a sample drawn from blood, serum, plasma, urine, sputum and saliva.

In another aspect of the present disclosure, there is provided at least one biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1) for the detection of oral cancer.

The present disclosure provides biomarkers for the diagnosis of oral cancer in an individual. The method comprises obtaining a sample from the individual and detecting the presence at least one biomarker identified as associated with oral cancer and the screening and analysis of the biomarker from the sample. Typically, the oral cancer can be diagnosed by determining and/or measuring the expression level of at least one biomarker as described in the present disclosure. Further, some of the proteins are down-regulated, whereas some proteins are up-regulated in oral cancer. These down-regulated or up-regulated proteins can also be determined using the method of the present disclosure.

The present disclosure is further described in light of the following experiments which are set forth for illustration purposes only and not to be construed for limiting the scope of the disclosure.

### EXPERIMENTAL DETAILS

### Experiment-1

A study was conducted using 20 oral cancer patient samples and 20 control cases of healthy individuals. A total of 53 potential biomarkers were identified with 25 proteins showing up-regulation and 28 proteins showing down-regulation in oral cancer patients. The main experimental steps of the study included serum/plasma dilution, biomarker assay, array washing after serum binding procedure, incubation with Cy3-anti IgG, washing after incubation with Cy3-anti IgG and data analysis (comprising the stages of cohort design, spot segmentation and statistical analysis), as given below.

Samples were placed in a shaking incubator set at 20° C. to allow thawing for 30 minutes. When the sample was completely thawed, each sample was vortexed vigorously three times at full speed and spun down for 3 minutes at 13,000 rpm using a microcentrifuge. 22.5 μL of the sample was pipetted into 4.5 mL of Serum Assay Buffer (SAB) containing 0.1% v/v Triton, 0.1% w/v BSA in PBS (20° C.) and vortexed to mix three times. The tube was tilted during aspiration to ensure that the sera was sampled from below the lipid layer at the top but does not touch the bottom of the tube in case of presence of any sediment. This Serum/Plasma dilution process was carried out in a class II Biological safety cabinet. Batch records were marked accordingly to ensure that the correct samples were added to the correct tubes. The array was removed from the storage buffer using forceps, placed in the slide box and rack containing 200 mL cold SAB and shaken on an orbital shaker at 50 rpm, for 5 minutes. When the slides have completed washing, the slide was placed, array side up, in a slide hybridization chamber with individual sera which had been diluted earlier. All slides were scanned using the barcode scanner into the relevant batch record and incubated on a horizontal shaker at 50 rpm for 2 hours at 20° C. The protein array slide was then rinsed twice in individual Pap jars with 30 mL SAB, followed by 200 mL of SAB buffer in the slide staining box for 20 minutes on the shaker at 50 rpm at room temperature. Binding of IgG was detected by incubation with Cy3Urabbit antiUhuman IgG (Dako Cytomation) labelled according to the manufacturer's recommended protocols (GE Healthcare). Arrays were immersed in hybridization solution containing a mixture of Cy3U rabbit antihuman IgG solution diluted 1:1000 in SAB buffer for 2 hours at 50 rpm in 20° C. After incubation, the slide was dipped in 200 mL of SAB buffer, 3 times for 5 minutes at 50 rpm at room temperature. Excess buffer was removed by immersing the slide in 200 mL of pure water for a few minutes. Slides were then dried for 2 minutes at 240 g at room temperature. Slides were then stored at room temperature until scanning (preferably the same day). Hybridization signals were measured with a microarray laser scanner (Agilent Scanner) at 10 μm resolution. Fluorescence levels were detected according to the manufacturer's instructions. Data sorting and analysis are done by customized computer scripts written using a Linux operating system, whereas each spot is plotted using Agilent Feature Extraction software. The potential biomarkers were identified and ranked using two methods:

1) penetrance-based threshold; and

2) volcano plot analysis.

Identification and ranking of protein biomarkers were done using a penetrance-based fold change. A penetrance-based fold change measures the likelihood that a given raw fold change is true, thus increasing the significance and reliability of the results.

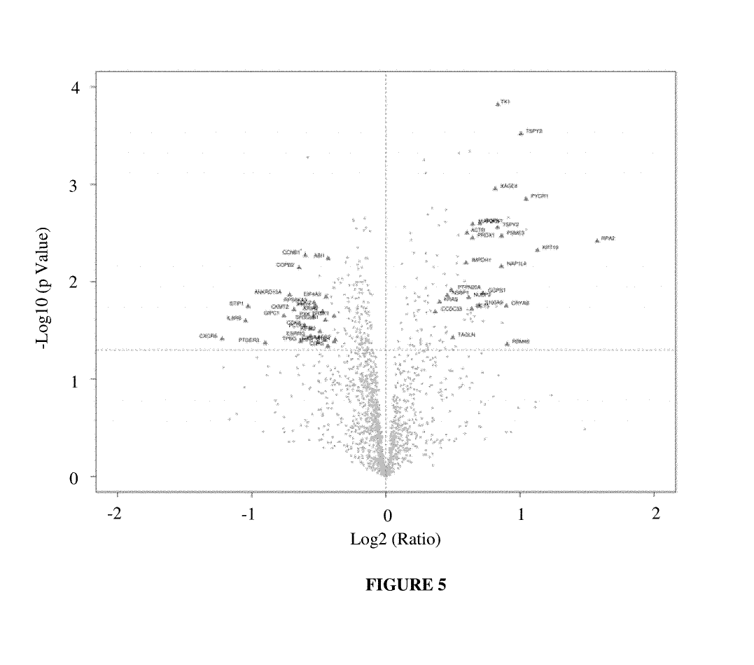

The volcano plot analysis was used for the identification of putative biomarker candidates and for plotting the Log 2 (overall fold change) on the x-axis versus−Log 10 (p-value) on the y-axis, as illustrated in FIG. 5.

FIG. 5 illustrates the Volcano Plot of the up-regulated and down-regulated biomarkers using penetrance-based analysis; wherein X-axis depicts the overall fold change and the Y-axis depicts the p-Value of the biomarkers.

Calculation of the overall fold change was calculated by dividing the mean of each protein across all case samples, μ(Hcase) with the mean of each protein across all control samples, μ(Hcontrol).

Volcano plot (FIG. 5) clearly depicts both the up-regulated and the down-regulated biomarkers identified using the penetrance-based analysis. The volcano plot shows a slight differential regulation of the potential biomarkers. The up-regulated proteins in case are located in the upper right side of the volcano plot and down-regulated proteins are located in upper left part of the plot.

The methods were used for comparison between the oral cancer patients and healthy patients. The potential biomarkers were identified and ranked according to the following criteria:
P-value ≤0.05;
For up-regulated biomarkers, Penetrance Fold Change Difference [i.e. (Penetrance Fold Change)Case−(Penetrance Fold Change)Control] was ≥2 and Frequency Differential ≥1;
For down-regulated biomarkers, Penetrance Fold Change Difference [i.e. (Penetrance Fold Change)Case−(Penetrance Fold Change)Control] was ≤−2 and Frequency Differential ≥−1;
Frequency percentage in case (i.e. Frequency case/Number of case×100%) must be ≥10%; and
Frequency percentage in control (i.e. Frequency control/Number of control×100%) must be ≥10%.

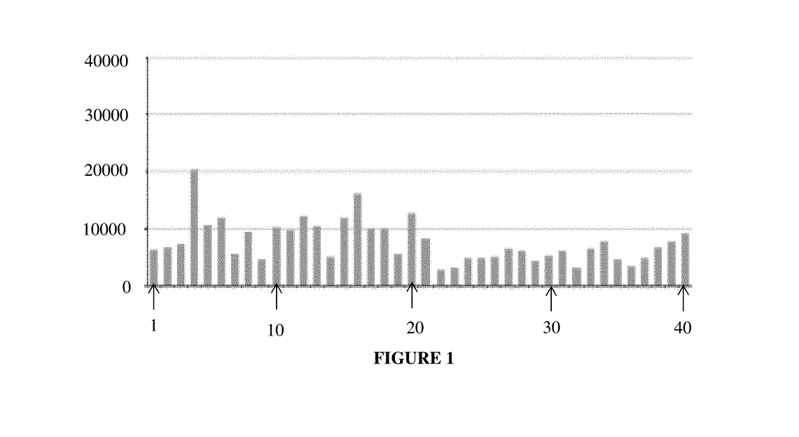

FIGS. 1 to 4 show increased expression of proteins in oral cancer cases as compared to controls suggesting that these proteins could serve as potential biomarkers for oral cancer.

FIG. 1 illustrates relative expression (up-regulation) of Thymidine kinase 1 (TK1) across oral cancer (1-20) and healthy control (21-40) samples; wherein X-axis depicts number of patient samples and Y-axis depicts normalized Relative Fluorescence Units (RFU) values.

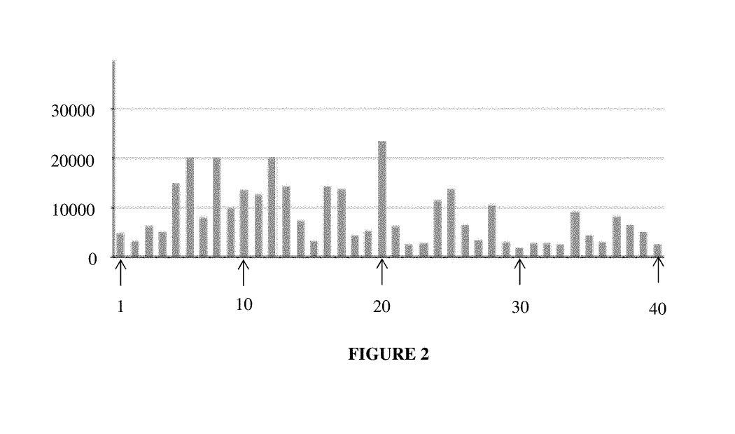

FIG. 2 illustrates relative expression (up-regulation) of PYCR1 (pyrroline-5-carboxylate reductase 1) across oral cancer (1-20) and healthy control (21-40) samples; wherein X-axis depicts number of patient samples and Y-axis depicts normalized Relative Fluorescence Units (RFU) values.

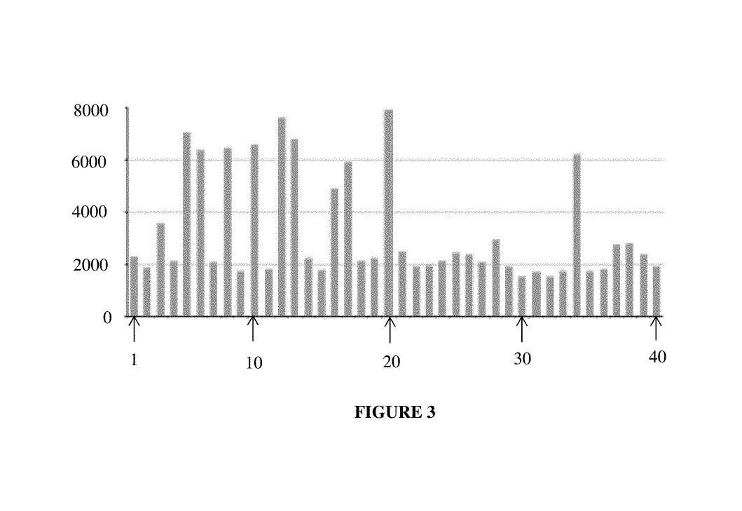

FIG. 3 illustrates relative expression (up-regulation) of Proteasome activator subunit 3 (PSME3) across oral cancer (1-20) and healthy control (21-40) samples; wherein X-axis depicts number of patient samples and Y-axis depicts normalized Relative Fluorescence Units (RFU) values.

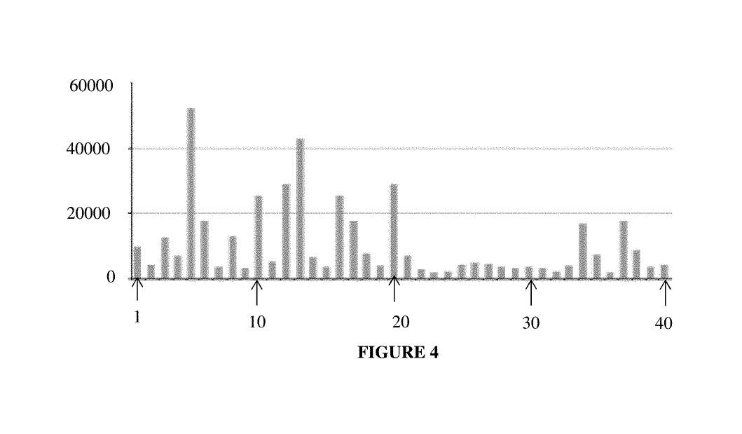

FIG. 4 illustrates relative expression (up-regulation) of Replication protein A2 (RPA2) across oral cancer (1-20) and healthy control (21-40) samples; wherein X-axis depicts number of patient samples and Y-axis depicts normalized Relative Fluorescence Units (RFU) values.

Thus, a total of 53 potential biomarkers were identified of which 25 showed up-regulation and 28 proteins showed down-regulation in oral cancer patients.

The embodiments as described herein above, and various features and advantageous details thereof are explained with reference to the non-limiting embodiments in the description. Descriptions of well-known aspects, components and molecular biology techniques are omitted so as to not unnecessarily obscure the embodiments herein.

The foregoing description of specific embodiments so fully reveal the general nature of the embodiments herein, that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein. Further, it is to be distinctly understood that the foregoing descriptive matter is to be interpreted merely as illustrative of the disclosure and not as a limitation.

Having described and illustrated the principles of the present disclosure with reference to the described embodiments, it will be recognized that the described embodiments can be modified in arrangement and detail without departing from the scope of such principles.

While considerable emphasis has been placed herein on the particular features of this disclosure, it will be appreciated that various modifications can be made, and that many changes can be made in the preferred embodiment without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

### TECHNICAL ADVANCEMENTS

The present disclosure described herein above has several technical advantages including, but not limited to, the realization of determination of the expression level of a biomarker in a sample. The expression level of the biomarker in a sample is correlated with the diagnosis of oral cancer.

The embodiments as described herein above, and various features and advantageous details thereof are explained with reference to the non-limiting embodiments in the description. Descriptions of well-known aspects, components and molecular biology techniques are omitted so as to not unnecessarily obscure the embodiments herein.

The foregoing description of specific embodiments so fully reveal the general nature of the embodiments herein, that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein. Further, it is to be distinctly understood that the foregoing descriptive matter is to be interpreted merely as illustrative of the disclosure and not as a limitation.

Having described and illustrated the principles of the present disclosure with reference to the described embodiments, it will be recognized that the described embodiments can be modified in arrangement and detail without departing from the scope of such principles.

While considerable emphasis has been placed herein on the particular features of this disclosure, it will be appreciated that various modifications can be made, and that many changes can be made in the preferred embodiment without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A method for detecting at least one biomarker in a sample, said method comprising the following steps:
a. diluting said sample containing said at least biomarker with serum albumin buffer to obtain a diluted sample;
b. contacting said diluted sample with a reagent comprising a set of capture antibody component capable of binding with said at least one biomarker in a one to one, one to many, and many to many correspondence, and at least one detection antibody component capable of detecting the binding of said capture antibody component with said biomarker; and
c. analyzing the change in the intensity of the detection antibody component to detect the presence of said biomarker and the extent of its expression,
wherein said at least one biomarker is selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

2. The method as claimed in claim 1, wherein said set of capture antibody component comprises antibody against said at least one biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1).

3. The method as claimed in claim 1, wherein said sample is serum.

4. The method as claimed in claim 1 comprises correlating the binding of the capture antibody component with said at least one biomarker in the sample with diagnosis of oral cancer.

5. At least one biomarker selected from the group consisting of Nucleotide binding protein 2 (NUBP2), Testis specific protein Y-linked 3 (TSPY3), XAGE-4 protein (XAGE4), Testis specific protein Y-linked 2 (TSPY2), Replication protein A2 (RPA2), Pyrroline-5-carboxylate reductase 1 (PYCR1), Thymidine kinase 1 (TK1), High mobility group nucleosome binding domain 5 (HMGN5/NSBP1), Rhophilin associated protein 1 (ROPN1, Ropporin), RNA binding motif protein 46 (RBM46), Proteasome (prosome, macropain) activator subunit 3 (PSME3), Keratin 19 (KRT19), Transgelin (TAGLN), Peroxiredoxin (PRDX1), S100 Calcium binding protein A9 (S100A9), and Inosine monophosphate dehydrogenase 1 (IMPDH1),
wherein said biomarker is used for the detection of oral cancer.
